(19) United States
(12) Patent Application Publication
(10) Pub. No.: US 2014/0180377 A1
(43) Pub. Date: Jun. 26, 2014

(54) ANEURYSM OCCLUSION SYSTEM AND METHOD

(71) Applicants: Arani Bose, New York, NY (US); Vikas Gupta, San Leandro, CA (US)

(72) Inventors: Arani Bose, New York, NY (US); Vikas Gupta, San Leandro, CA (US)

(73) Assignee: Penumbra, Inc., Alameda, CA (US)

(21) Appl. No.: 13/722,709

(22) Filed: Dec. 20, 2012

(51) Int. Cl. A61F 2/07

(52) U.S. Cl. CPC A61F 2/07
USPC 623/1.11

(57) ABSTRACT

An aneurysm occlusion system includes devices positionable within a cerebral blood vessel covering a neck of an aneurysm in the blood vessel. A component device includes an expandable tubular body, an expandable anchor, and a link connecting the body to the anchor. One or more devices deployed using a method according to the invention includes a novel feature that guarantees that the distal high coverage segment aligns with the neck of the aneurysm. A single device or multiple devices, used in conjunction with an embolic material or coil, may be combined to form a system according to the invention. When positioned and deployed strategically either alone or with a second device utilizing a method according to the invention, the system has a high coverage region covering a neck of an aneurysm, and a gap between the system and healthy vessel. The system and method prevent blood flow into an aneurysm while permitting blood flow through healthy vessel.

### BACKGROUND OF THE INVENTION

### Field of the Invention

The present invention relates generally to the fields of systems and methods for treatment of an aneurysm in a blood vessel, including implanting one or more intravascular devices for occlusion of the aneurysm. The invention herein is especially useful in treating an aneurysm located at or near a vessel bifurcation.

An aneurysm is an abnormal ballooning of a portion of a blood vessel wall due to weakening of the wall tissue. While aneurysms can occur in any artery of the body, a large percentage of aneurysms are found in the cerebral blood vessels. An aneurysm may develop at or near a bifurcation, where a main vessel branches into two or more separate vessels, and thereby present unique challenges for successful treatment. If left untreated, an aneurysm can rupture, leading to life threatening hemorrhaging in the brain which can result in death or severe deficit. Aneurysms that do not rupture can form blood clots which can break away from the aneurysm, potentially causing a stroke. In some patients, aneurysm can put pressure on nerves or brain tissue, causing pain, abnormal sensations, and/or seizures.

Numerous devices and methods for the treatment of aneurysm are in use and many more are in development. An example of a current practice for treatment of an aneurysm includes surgical placement of an aneurysm clip across the aneurysm to prevent blood flow into the aneurysm. Naturally, this procedure requires highly invasive brain surgery and thus carries many risks.

In a less invasive, catheter-based technique for aneurysm treatment, an aneurysm may be packed with a filler material in order to block blood flow into the aneurysm. Such filler material is carried through the vasculature and to the site of the aneurysm which is then selectively filled. Materials used for this purpose include platinum coils and cellulose acetate polymer to fill the aneurysm sac. While these techniques have had some success, questions remain concerning their long-term effectiveness, ease of use, and their potential for rupturing the aneurysm or triggering clot formation. In addition, there is some risk of post procedure migration of embolic material from the aneurysm into the parent blood vessel.

According to another prior art aneurysm treatment, a mesh or braided stent-like device is positioned within a blood vessel such that it bridges the aneurysm, thereby preventing migration of embolic material out of the aneurysm following implantation. A problem is encountered with devices of this type is when the sidewalls of the device block flow between the blood vessel and any side branch vessels that the stent cover incidentally. Another potential problem with devices of this type is the difficulty of positioning the devices within a vessel, especially if the aneurysm is located at or near a bifurcation in the vessel.

A schematic drawing of an aneurysm within a branched or bifurcated vessel is in FIG. 1A. FIG. 1A illustrates aneurysm A, located along branch vessel B, near bifurcation F. Blood flows through vessel V generally in the direction of arrow D1. At bifurcation F, vessel V bifurcates to form branch vessel B. Branch vessel B includes left branch vessel L and right branch vessel R. At bifurcation F, blood flows in the direction of D2 in left branch vessel L, and in the direction of D3 in right branch vessel R. However, blood also flows into aneurysm A and within aneurysm A in a multitude of directions. As described above, blood flow into the aneurysm may lead to expansion and eventual rupture of aneurysm A. Therefore, it is desirable to prevent blood flow into aneurysm A, yet permit blood flow through branch vessel B.

Neck N defines the “entrance” to aneurysm A. Neck N is very near bifurcation F, and extends from left branch vessel L to right branch vessel R. In general among aneurysms, the width of a neck varies. In the example of aneurysm A, neck N has a significant width. Therefore, a substantial risk of post implantation migration of embolic materials through neck N, out of aneurysm A, and into vessel V exists, as is apparent in FIG. 1A. Migration of a coil or other embolic material into the circulatory system is undesirable and can lead to occlusion of the parent vessel and/or other vessels, as well as lead to other unintended effects. Moreover, the challenge of effectively covering neck N, without blocking blood flow into and through healthy vessel V and healthy portions of left branch vessel L and right branch vessel R, is clear in FIG. 1A.

An example of an embolic coil suitable for implantation into an aneurysm using minimally invasive techniques is described fully in U.S. patent application Ser. No. 12/498,752. Other embolic coils that suitable for use with the present invention are known and used in the art. Regardless of the particular embolic material implanted into aneurysm A, the risk of migration of such materials out of aneurysm A and into vessel V is undesirable. An example of such a migration is illustrated in FIG. 1B. FIG. 1B illustrates a vessel such as vessel V following implantation of coil C. An end of coil C has begun to migrate out of aneurysm A and into right branch vessel R. It is desirable to place a bridge device across neck N to prevent such a migration. However, there are difficulties in effectively deploying a bridge device in a vessel having the configuration of vessel V. Further, as is also evident in FIGS. 1A and 1B, there are challenges in effectively covering neck N in order to prevent migration of embolic material, or to divert blood flow from the interior of aneurysm A, or both, without blocking blood flow into and through healthy vessel V.

Shortcomings of prior art attempts to bridge the neck of an aneurysm located at or near a bifurcation include failure to achieve sufficient coverage of the neck of the aneurysm; failure to avoid blockage of block blood flow to healthy vessel; insufficient accommodation of variation in vessel and aneurysm structure; unreliable tracking and deployment of the device; unacceptable levels of difficulty with resheathing and repositioning the device when needed; and irregular or inconsistent deployment geometry of the device adjacent the aneurysm. Therefore, there remains a need for sufficient aneurysm neck coverage without sacrificing blood flow to healthy vessel; for a system which can accommodate variations in vessel anatomy; for smooth, kink-free tracking, deployment, repositioning, and reliable, uniform deployment. There also remains a need, both for delivery and for accommodating vessel pulsation, for a sufficiently flexible device having adequate column strength.

### BRIEF DESCRIPTION OF THE DRAWINGS

FIG. 1A schematically illustrates blood flow into an aneurysm located near a bifurcation in a blood vessel.

FIG. 1B illustrates an early stage of migration of an embolic coil following implantation into aneurysm A.

FIG. 2A illustrates a device according to the invention deployed within a vessel having an aneurysm near a bifurcation of the vessel, and the resulting prevention of migration of coil C.

FIG. 2B illustrates an alternative embodiment of a system according to the invention, the system deployed within a vessel having an aneurysm near a bifurcation of the vessel.

FIG. 2C illustrates detail E of FIG. 2B.

FIG. 3A illustrates a side view of devices according to the invention, where the devices are deployed outside a vessel.

FIG. 3B illustrates detail G of FIG. 3A.

FIG. 3C illustrates an alternate view of detail G of FIG. 3A.

FIG. 4A is a perspective view of a single finished device according to the invention, the device in a deployed configuration outside a vessel.

FIG. 4B is a bottom view of the single finished device of FIG. 4A.

FIG. 5 illustrates a plan view of a cut pattern for a device according to the invention. Although devices according to the invention are preferably cut from tubular structures and shape set, FIG. 8 illustrates the cut tube as though it were longitudinally cut and flattened into a sheet, so that the pattern features may be more easily viewed.

FIGS. 6A through FIG. 6F illustrate steps of a method of treatment according to the invention.

FIGS. 7A through 7B illustrate steps of an alternative method of treatment according to the invention.

FIGS. 8A through 8D illustrate examples of alternate set shapes of a devices according to the invention, where the devices are shown in their deployed configuration outside of a vessel.

### DETAILED DESCRIPTION

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be routine undertaking for those of ordinary skill in the art having the benefit of this disclosure. The aneurysm occlusion system disclosed herein exhibits a variety of inventive features and components that warrant patent protection, both individually and in combination.

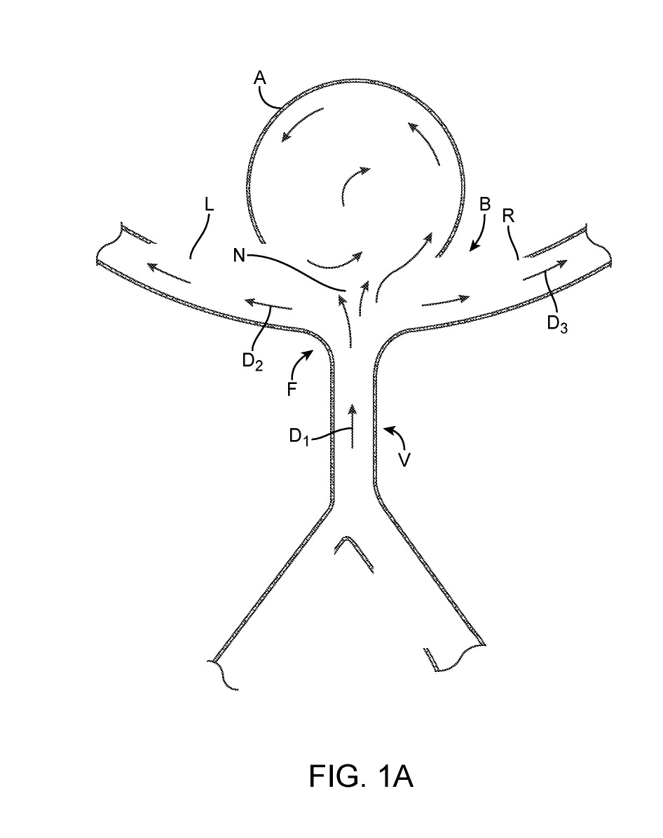

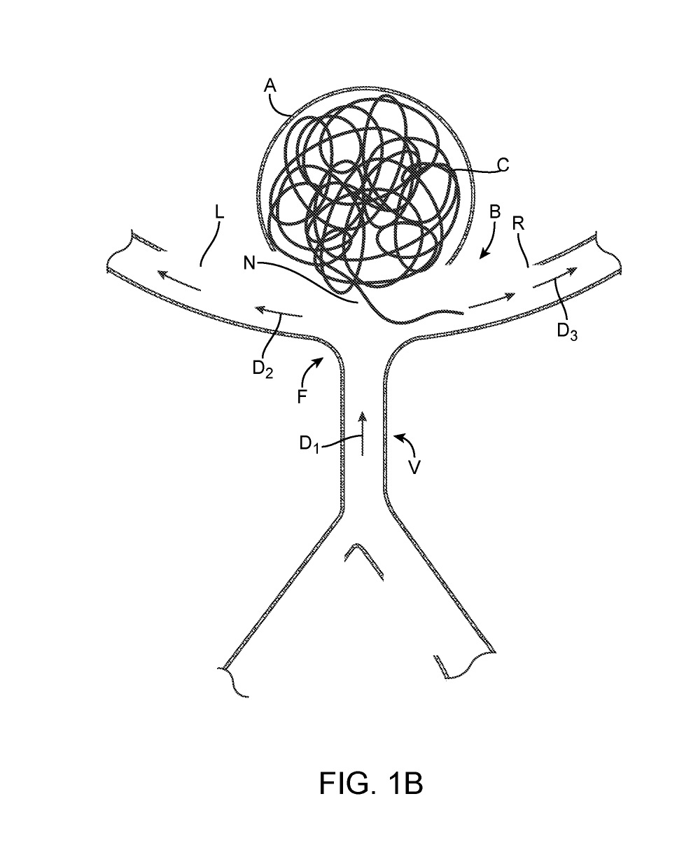

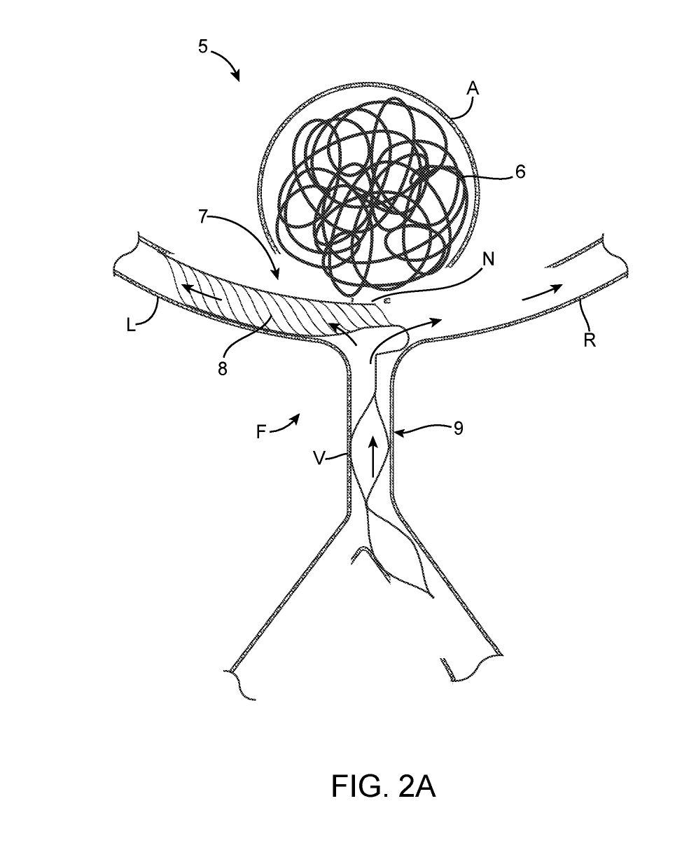

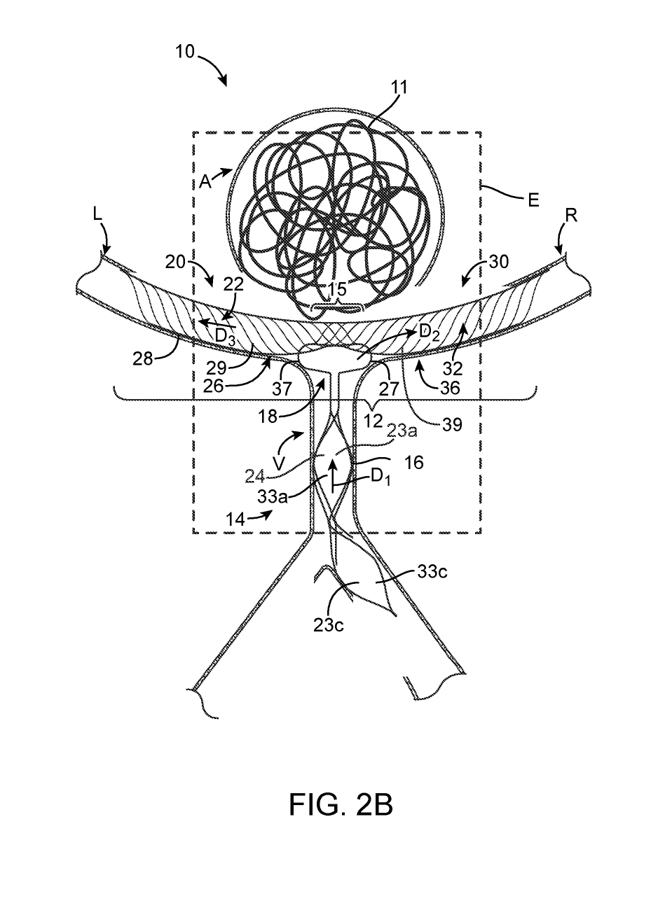

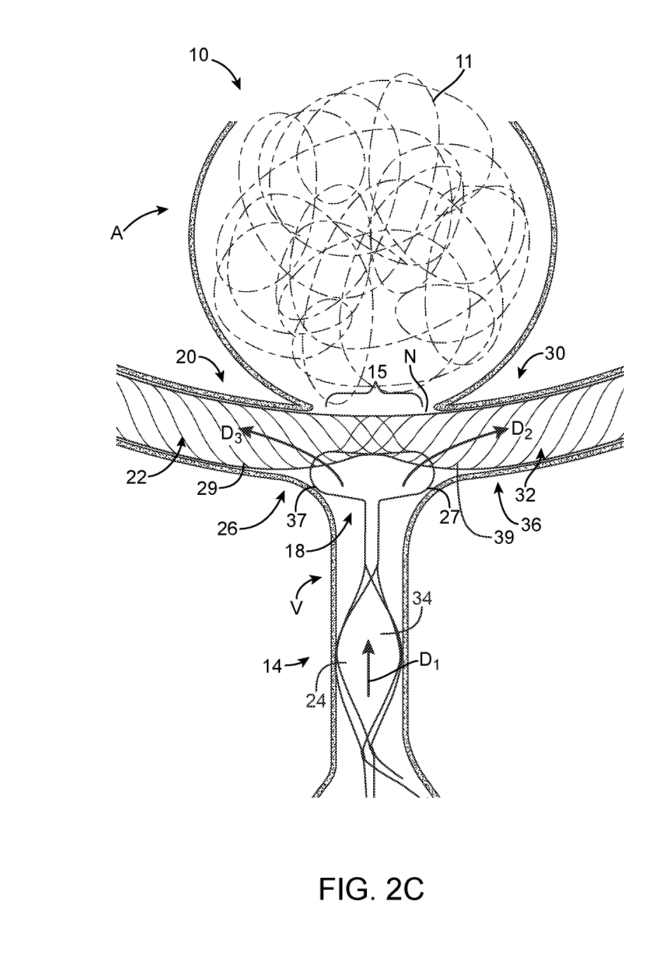

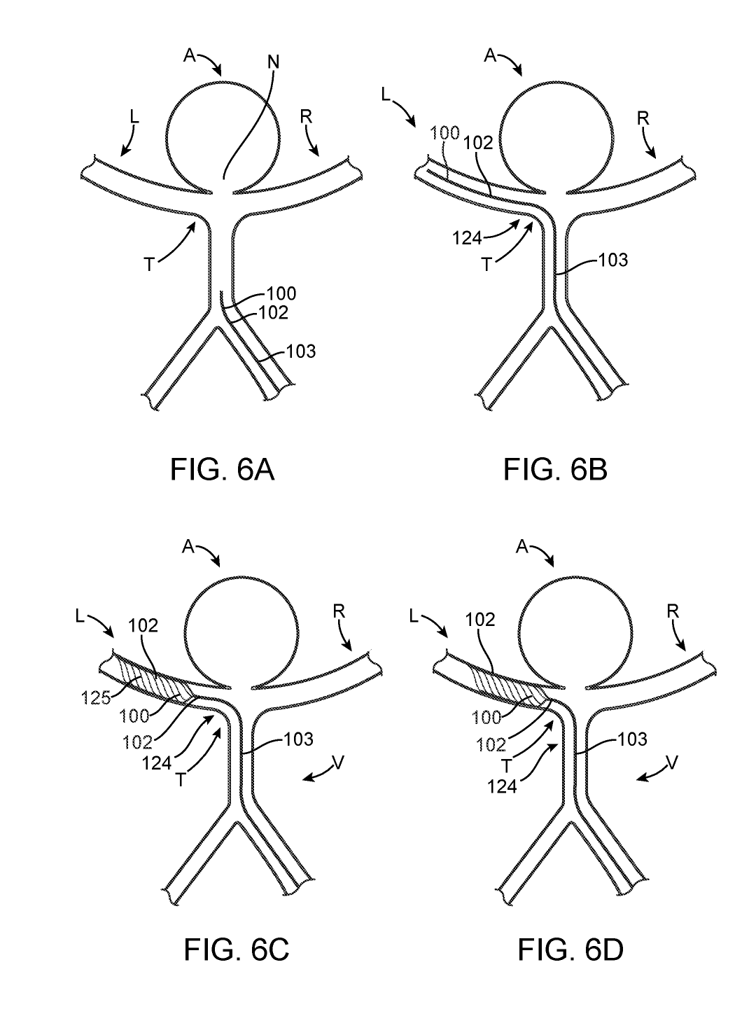

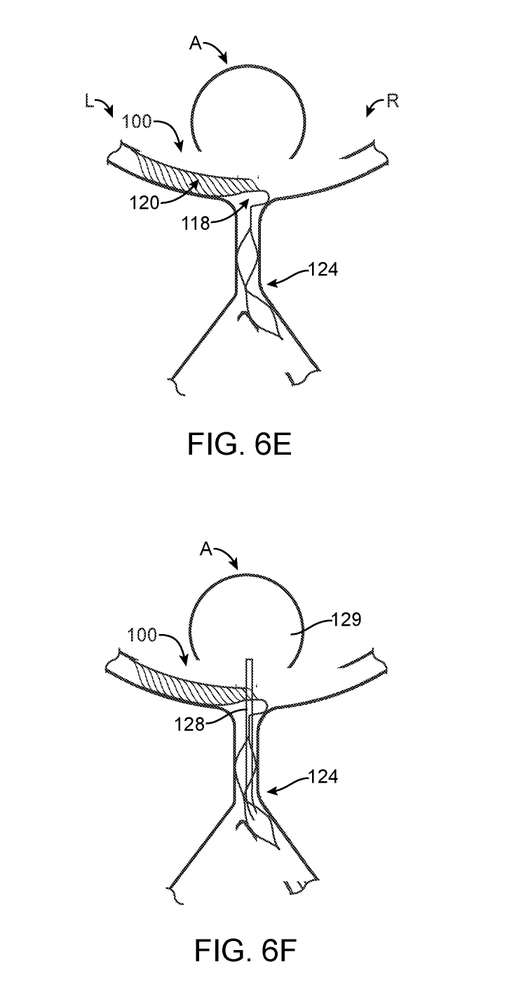

FIG. 2A illustrates a system according to the invention deployed in a vessel that is similar to the vessel illustrated in FIG. 1. In vessel V, aneurysm A is located near bifurcation F. Vessel V bifurcates at bifurcation F to form left branch vessel L and right branch vessel R. System 5 is illustrated in its deployed, expanded profile configuration, with a component near aneurysm A and another component within aneurysm A. Generally speaking, system 5 includes a coil 6 and a bridge 7. Bridge 7 is an intravascular device that has a span 8 and an anchor 9. Bridge span 8 is deployed and in its final position lies mostly within left branch vessel L and across aneurysm neck N. Bridge 7 can thereby prevent migration of embolic coil 6 out of aneurysm A. Bridge 7 is the same as or similar to the intravascular devices which are described in greater detail below. Anchor 9 is deployed mostly within vessel V and functions to securely maintain the desired position of bridge 7. The steps by which bridge 7 is delivered and deployed, and examples of steps by which embolic coil 6 may be implanted into aneurysm A are described more fully below and illustrated in FIGS. 6A-6F. Though the example of FIG. 2A includes embolic coil 6, other embolic structures or materials may be suitable for use within a system according to the invention.

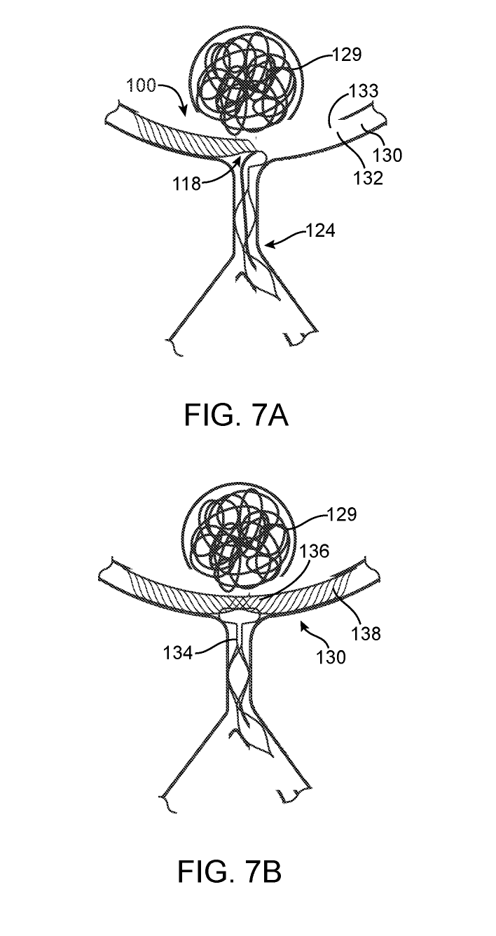

An alternative embodiment of the invention is illustrated in FIG. 2B. System 10 is illustrated in FIG. 2B in its deployed, expanded profile configuration both within and near aneurysm A. Generally speaking, system 10 includes coil 11 deployed within aneurysm A, and two intravascular devices that together form T-span 12 and anchor 14. T-span 12 is disposed in left branch vessel L, across neck N of aneurysm A, and into right branch vessel R. T-span 12 includes high coverage region 15, which spans much or all of neck N, and also prevents migration of coil 11 out of aneurysm A. Anchor 14 is disposed within vessel V. Lateral apexes 16 of anchor 14 function to securely position anchor 14 within vessel V, and to secure system 10 within the vessel. The steps by which the intravascular devices of system 10 may be delivered and deployed are discussed below and illustrated in FIGS. 6A-7B.

FIG. 2B also illustrates schematically the direction of blood flow resulting from deployment of system 10. Blood continues to flow from vessel V to branch vessel B in the direction of arrow D1. That is, blood is permitted to flow through the generally open interior of anchor 14. Blood also continues to flow through right branch vessel R in the direction of arrow D2. And blood continues to flow through left branch vessel L in the direction of arrow D3. However, blood no longer flows into aneurysm A, as it previously had in a manner such as that illustrated in FIG. 1A above. Blood is prevented by system 10 from flowing into aneurysm A by coil 11 and by high coverage region 15. Within high coverage region 15, there are a plurality of bands or struts (the particular configurations of which are discussed in detail below) that prevent migration of coil 11, and also prevent blood flow therethrough.

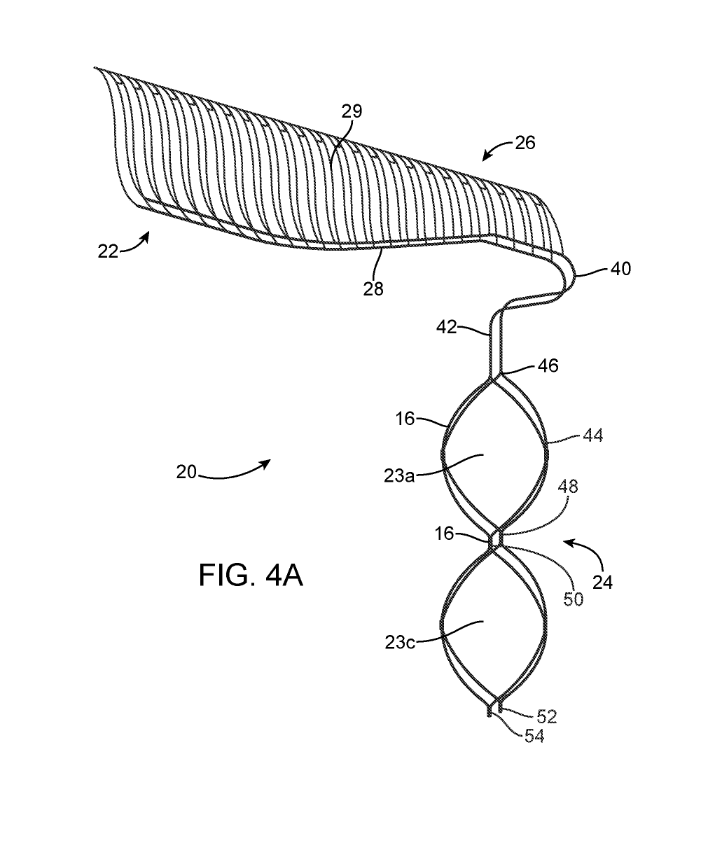

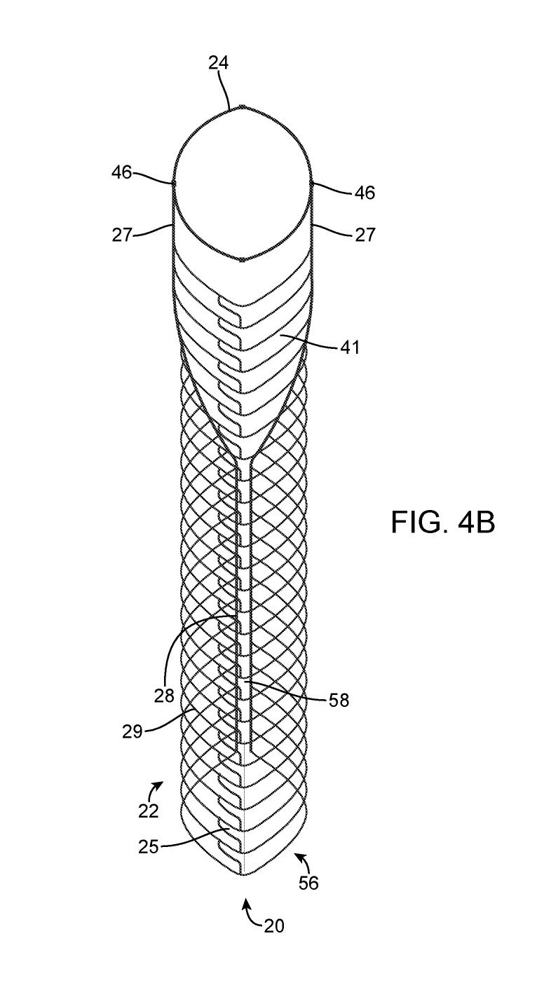

Though it is difficult to see in FIG. 2B, opposite high coverage region 15 of system 10 is open side 18. There are no struts, or very few struts, in open side 18. (The specific configuration of open side 18 is more readily discernible in FIG. 4B below and its related text.) Open side 18 permits blood flow between vessel V and branch vessel B in the direction of arrow D1. Open side 18 also permits blood to continue to flow through branch vessel B in the directions of arrows D2 and D3.

T-span 12 is defined by two generally tubular segments of the component intravascular devices that are referred to as first bridge 20 and second bridge 30. (First bridge 20 and second bridge 30 are the same as, or similar to, bridge 7 of FIG. 2A above.) In their deployed, expanded profiles, both first bridge 20 and second bridge 30 have generally hollow interiors through which blood flow is permitted. (First bridge 20 and second bridge 30 also each have a reduced profile, delivery configuration, for use in a method which will be illustrated and described below.) First bridge 20 and second bridge 30 are proportioned to be implanted within the cerebral vasculature including, but not limited to, the Internal Carotid Artery, External Carotid Artery, Vertebral Artery, Basilar Artery, Middle Cerebral Artery, Anterior Cerebral Artery, and the Posterior Cerebral Artery. Preferred devices are expandable to an outer diameter in the range of 1.5 mm-6.0 mm. The user may be provided with a set of multiple devices of different diameters and different lengths so that the device with the most appropriate dimensions may be chosen for the procedure.

Suitable materials for first bridge 20 and second bridge 30 include shape memory materials such as, for example, superelastic Nitinol or shape memory polymers, other materials such as stainless steel, composite materials, or combinations of metals and polymeric materials, or from any number of compositions having suitable biocompatibility and strength characteristics. In a preferred embodiment, first bridge 20 and second bridge 30 are constructed from Nitinol® with “shape memory” or superelastic characteristics to optimize self expansion of the device upon deployment.

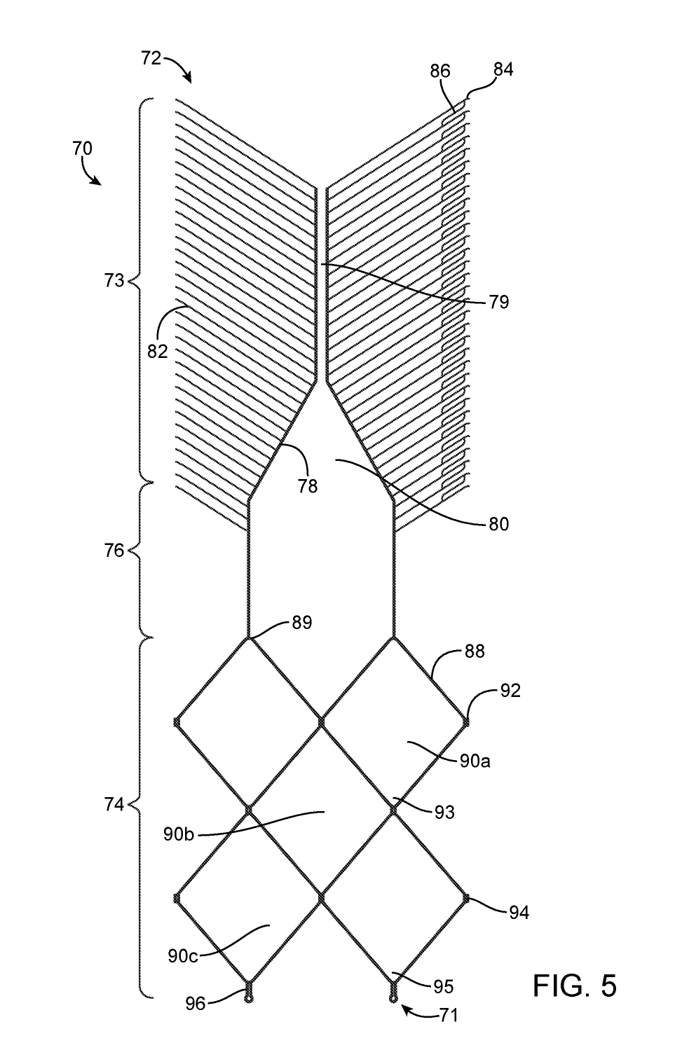

First bridge 20 and second bridge 30 are formed by laser cutting features into a length of superelastic Nitinol tubing. For example, a tube of 3.5 mm outer diameter and 0.005 inch thickness may be cut in a predetermined pattern of bands, standards, bands, struts, and/or connectors. An example of a suitable pattern is illustrated in detail in FIG. 5, though variations on the cut patterns and the sizes of nitinol tubing are within the scope of the invention. The cut tube may then be chemically processed and shape-set one or more time using methods known to those skilled in the art. The device may then be chilled to below its shape memory transition temperature (martensitic phase) and loaded onto a pusher and retained by a sheath, or other suitable delivery device (not pictured).

The primary features of first bridge 20 include body 22, proximal anchor 24, and links 27. Links 27 connect body 22 to proximal anchor 24. According to the invention first bridge 20 may be dimensioned in any number of suitable sizes and lengths, depending upon the location of the aneurysm, variances in patient anatomy, and the size and shape of the aneurysm. In its expanded configuration, first bridge 20 is approximately 1.5-6.0 mm at its maximum outer diameter, and between 6-50 mm in length.

Following the construction of coil 11, first bridge 20 and second bridge 30, system 10 is created by the strategic positioning and deployment of coil 11, first bridge 20 and second bridge 30. When first bridge 20 is in its deployed configuration, the interior of body 22 is generally hollow. The “walls” of body 22 are generally defined by standards 28 and bands or struts 29 that will be described in further detail below. Much of the exterior “walls” of body 22, in the form of standards 28 and struts 29 fit in close apposition with the walls of left branch vessel L. (Exceptions lie in the areas of split 21 and gap 25, which will be illustrated and described more specifically in FIG. 4B, and which define part of open side 18 of system 10 mentioned above.) Lateral apexes 16 of cells 23a, 23b and 23c of proximal anchor 24 extend radially outwardly from a central longitudinal axis of system 10, and are then in close apposition with the walls of vessel V.

In the example of FIG. 2B, second bridge device 30 is essentially of the same structure as first bridge 20. Therefore, second device 30 includes body 32, connected to anchor 34 by links 37. Also similar to body 22, in its deployment configuration, body 32 has a generally hollow interior. In addition, most of the “walls” of body 32 fit in close apposition with the walls of right branch vessel R. Second device 30 also has a proximal anchor 34 that is deployed within vessel V. Proximal anchor 34 includes cells 33a, 33b and 33c, and is shown deployed more or less within proximal anchor 24, via a method described in detail below in a description of FIGS. 7A-7B. Moreover, second bridge 30 is deployed so that some portion of base 26 of body 22 overlaps second device 30. Specifically, in this example, some portion of base 26 overlaps some portion of base 36 of second device 30. This area of overlap of base 26 and base 36 results in the overlap of many of the bands or struts 29 and bands or struts 39, and consequently defines a region of greater strut density in a portion of the “wall” of system 10.

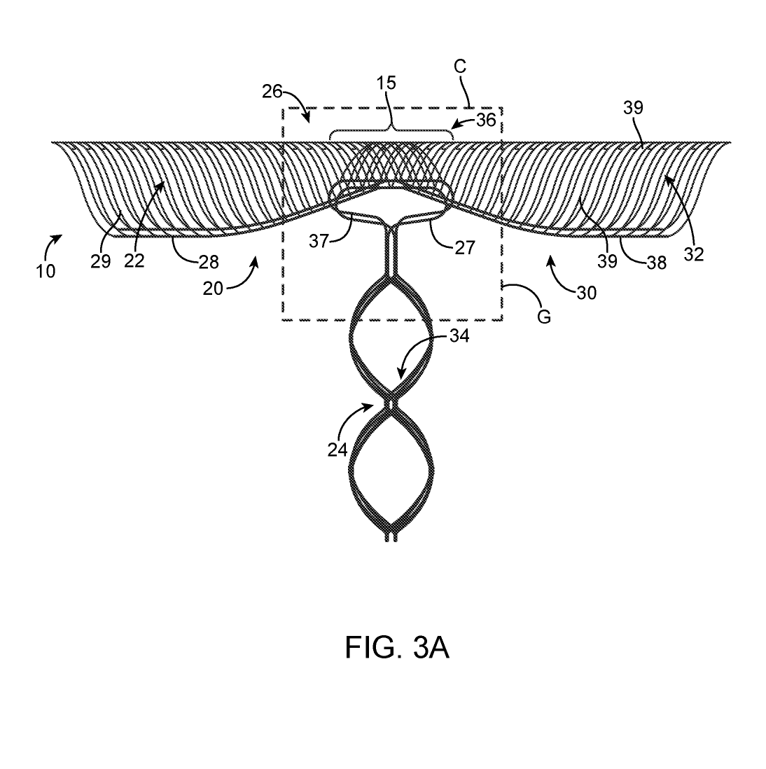

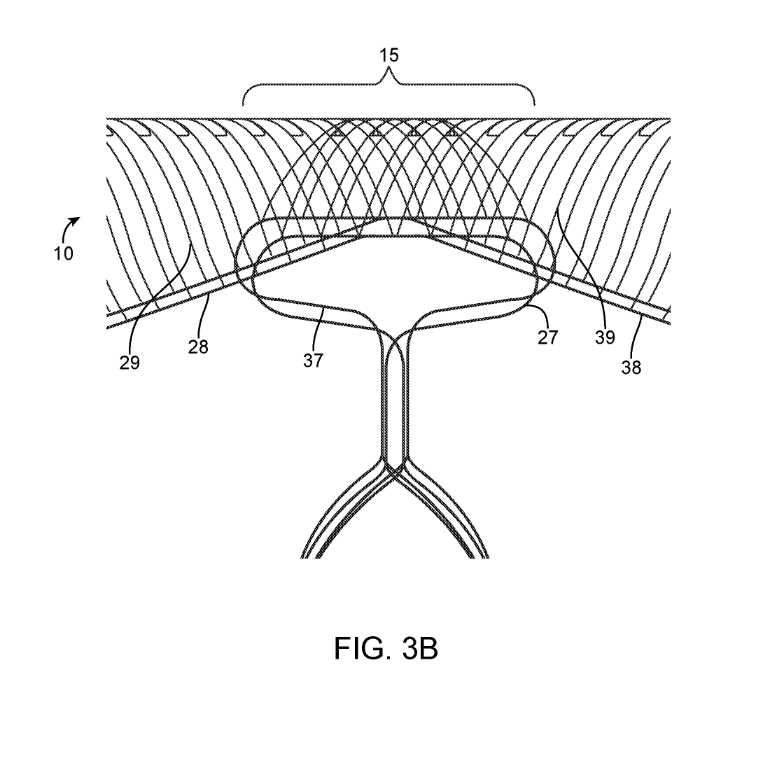

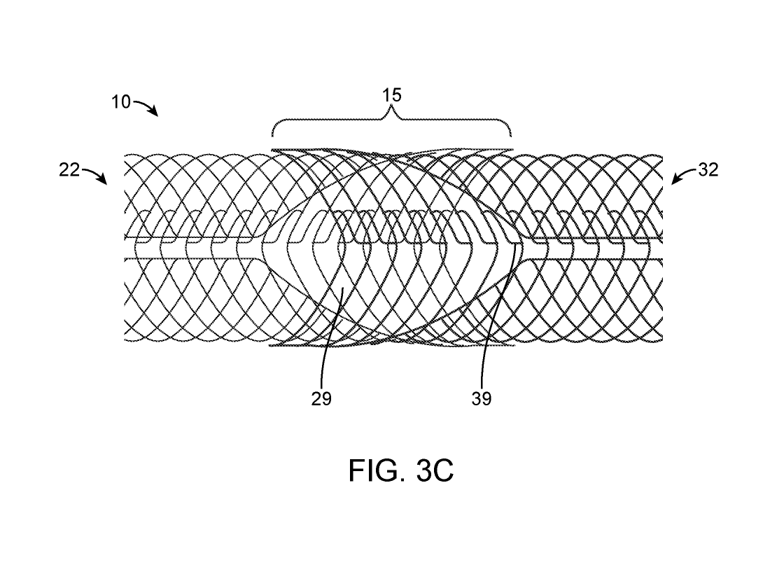

The area of greater concentration of struts 29 and 39 in the wall of system 10 is referred to as high coverage region 15. (In an alternative configuration to that shown in FIG. 2B, first device 20 and second device 30 could be deployed so that either a greater or a lesser portion of base 26 overlaps with base 36, in order to define a greater or lesser area of high coverage.) In the example of FIG. 2B, high coverage region 15 spans neck N of aneurysm A. High coverage region 15 provides robust support to the embolic coil filling the aneurysm, and/or prevents blood flow from entering aneurysm A. High coverage region 15 can be viewed in greater detail in FIG. 2C, which represents a closer view of vessel V at bifurcation F, and neck N of aneurysm A. High coverage region 15 may also be viewed in FIGS. 3A, 3B and 3C, which represent alternate perspectives of this feature of the system.

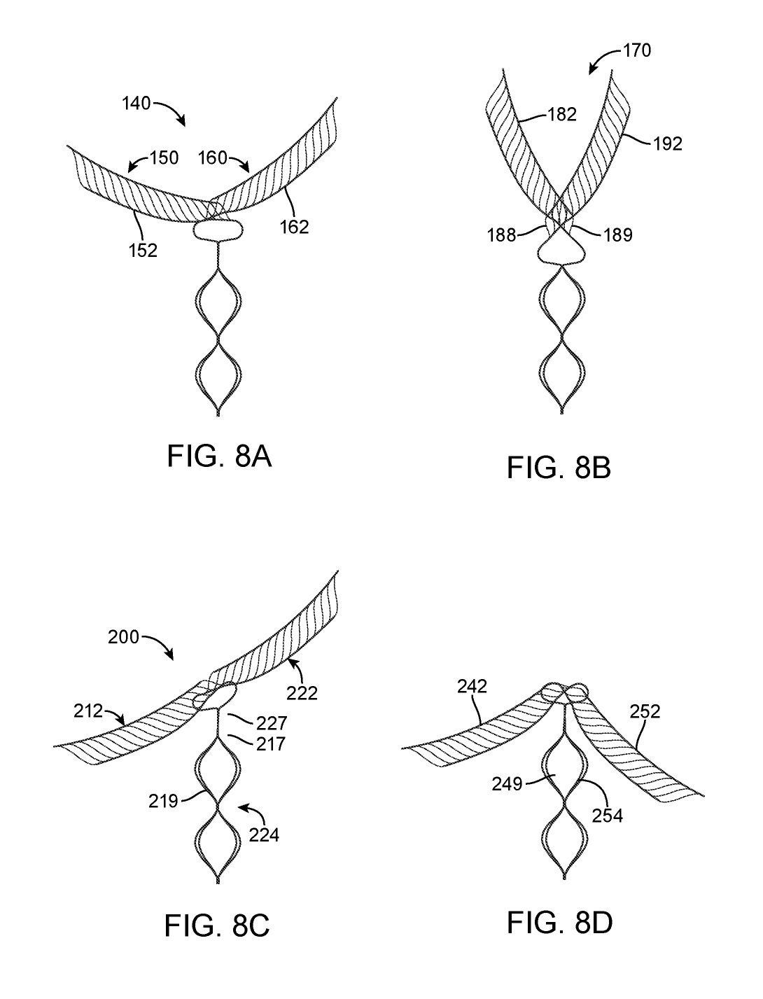

In FIG. 2B, first bridge device 20 and second bridge device 30 are essentially identical to one another, except for the placement of the devices within the vasculature prior to deployment, especially the positions of deployment of body 22 and body 32 and the resulting high coverage region 15. In alternative embodiments, first bridge device 20 and second bridge device 30 may differ from one another. For example, the body of a device may be shape set in an alternative configuration in relation to a proximal anchor in order to accommodate variances in vessel morphology and/or a different vessel and side branch configuration. Some examples of alternative shape sets are described below in relation to the discussion of FIGS. 8A-8D. In alternative embodiments, either or both first bridge device 20 and second bridge device 30 may be sized, shaped or shape set in alternative orientations, in order to accommodate variances in patient anatomy, vessel morphology and other factors.

FIG. 2C represents a closer look at a portion of system 10 of FIG. 2B, because it illustrates Detail area E. Consequently, some features of system 10 are more visible in FIG. 2C. Specifically, high coverage region 15 is shown spanning neck N of aneurysm A. A portion of anchor 14 is shown disposed within vessel V Links 27, which connect body 22 to proximal anchor 24, and links 37, which connect body 32 to proximal anchor 34 are also visible. Similarly, struts 29 of first bridge 20, and struts 39 of second bridge 30 are visible. FIG. 2C also illustrates schematically the direction of blood flow from vessel V to branch vessel B, and within branch vessel V near aneurysm A as a result of deployment of system 10. Instead of flowing into aneurysm A, as was schematically illustrated in FIG. 1A above, blood now flows through branch vessel B primarily in the directions represented by arrows D2 and D3. Blood is diverted by system 10, and most specifically coil and high coverage region 15, from flowing into aneurysm A.

Turning now to FIG. 3A, a portion of system 10 is illustrated in a deployed configuration outside of a vessel. The portion of system 10 represented in FIG. 3A includes first bridge 20 and second bridge 30. First bridge 20 and second bridge 30 are configured in FIG. 3A as they would be within a vessel, so that body 22 extends toward the left of links 27, and body portion 32 extends toward the right side of the figure. This portion of system 10 includes high coverage portion 15, defined by the overlap of struts 29 of base 26, with struts 39 of base 36. (High coverage portion 15 may alternatively be of greater or lesser area by varying the amount of overlap of base 26 and base 36.) In the view of FIG. 3A, proximal anchoring portion 24 coincides nearly completely with proximal anchoring portion 34. In alternative embodiments, either or both proximal anchoring portion 24 and proximal anchoring portion 34 may vary in size, shape, and/or amount of overlap when deployed.

FIG. 3B illustrates high coverage portion 15 of system 10 in detail. Beginning at the center of FIG. 3B, links 27 and links 37 are visible as occurring in pairs, though in previous figures only one each of a set of links 27 and links 37 were visible. Each link 27 extends distally to form longitudinal standard 28. And similarly, each link 37 extends distally to form longitudinal standard 38. Standards 28 and standards 38 have a width in the range of 0.0020-0.0050 inch, preferably between 0.0025-0.0045 inch, and impart axial and columnar strength upon first bridge 20 and second bridge 30. The standards may also be used to provide axial force to the device if it is necessary to reposition the device after partial deployment within the vessel as discussed above. Struts 29 extend from longitudinal standards 28. And extending from longitudinal standards 38 are struts 39. Struts 29 and 39 have strut widths in the range of 0.00050-0.00150 inch, and preferably between 0.00060-0.00120 inch. Struts 29 and struts 39 are typically separated from other struts 29 and struts 39 by a distance or gap of approximately 0.00060 inch to 0.0080 inch. Some of struts 29 overlap some of struts 39 to define high coverage portion 15. Longitudinal standards 28 and 38 and struts 29 and 39 are among the primary structures defining body 22 and body 32 shown in previous figures.

FIG. 3C illustrates a portion of system 10 as though the viewer were looking down on the “top” of this portion of the system. If the system 10 were deployed in a vessel, the view would be as though one were looking “down” the neck of an aneurysm. Centered in FIG. 3C is a top or plan view of high coverage portion 15. High coverage portion 15 is defined by the overlap of some of body 22 and some of body 32. (In a vessel, high coverage portion 15 would be positioned to span much or all of the neck of an aneurysm.) The overlap of struts 29 with struts 39 is visible in FIG. 3C. In addition, S-connectors 25 of body 22 and S-connectors 35 of body 32 are visible.

The components of first bridge 20 and second bridge 30 of system 10 (which may be similar to bridge device 7) will now be described in detail. Specifically, FIGS. 4A and 4B illustrate first bridge 20 in isolation from system 10. Because bridge 20 is illustrated separately from system 10, the details of first bridge device 20 can be more easily viewed. And because second bridge 30 (not pictured in FIGS. 4A and 4B) is substantially identical to first bridge 20, the features all of the devices can be more clearly understood.

FIG. 4A represents a side view of first device 20 in its deployed configuration. First device 20 includes body 22 having a base 26. Body 22 is defined primarily by longitudinal standards 28, and struts 29, which extend transversely from longitudinal standards 28. Longitudinal standards 28 extend proximally from the base 26 of body 22 to define links 27. Links 27 extend proximally from base 26 to connect body 22 to proximal anchor 24. Though other shapes are possible within the scope of the invention, each longitudinal standard 28 together with each link 27 roughly resembles the shape of the numeral “7”. The portions of longitudinal standards 28 near the base 26, combined with hairpin turns 40, form the top of the “7”. Proximally from hairpin turns 40, links 27 again curve sharply to define uprights 42, resembling the downward stroke of the numeral “7”.

At the beginning of the downward stroke of the “7”, each upright 42 splits or divides to form two tines 44 that roughly outline a “diamond” shape, referred to as cell 23a. Cell 23a has a top apex 46 and a bottom apex 48 where the tines 44 rejoin. Between top apex 46 and bottom apex 48 are lateral apexes 16 mentioned above in connection with the description of system 10. From bottom apex 48, cell 23a in turn extends proximally to connect to top apex 50 of cell 23c. Cell 23c also has a bottom apex 52. Oriented roughly at 90 degrees to cells 23a and 23c, and therefore not visible in the view of FIG. 4A, are cells 23b. Though the example of FIG. 4A includes three sets of cells 23a, 23b and 23c, a greater or lesser number is also within the scope of the invention. Further, the shape of cells 23a, 23b and 23c may be varied widely and include more or less elliptical lines or more or less straight and angled lines. Attached to bottom apex 52 are markers 54 for enhanced visualization. As mentioned above, second bridge 30 of system 10 has essentially identical features corresponding to those of first bridge 20.

FIG. 4B illustrates an alternative perspective view of first bridge device 20 of FIG. 4A. FIG. 4B is a view from the “bottom” of first bridge device 20. From this view, longitudinal standards 28 can be seen extending much of the length of body 22. Beginning at distal end 56, longitudinal standards 28 lie generally parallel to one another to define split 58. Longitudinal standards then diverge from one another to define gap 25. Longitudinal standards may diverge widely enough, and then descend to form links 27, and ultimately to be disposed directly opposite one another where they attach to top apexes 46.

Extending somewhat transversely from longitudinal standards 28, along the length of body 22, are struts 29. Bands or struts 29 further include S-connectors 41, which can be seen along the “wall” of body 22, generally opposite split 21. From body 22, longitudinal standards 28 extend proximally to define links 27. Links 27 connect body 22 to proximal anchor 24. The generally open interior of proximal anchor 24 is visible in FIG. 4B.

First bridge 20 of system 10 is preferably manufactured from a tube or comparable structure that is cut according to a predetermined pattern. FIG. 5 illustrates a cut pattern 70 used in the construction of a device such as first bridge 20. The pattern 70 of FIG. 5 is presented as though a tube cut according to the invention, prior to shape setting, were also cut longitudinally and then flattened so that the features of the cut tube are more easily seen. Pattern 70 includes a proximal end 71 and a distal end 72, and generally speaking, three regions therebetween. Body segment 73 is disposed near distal end 72, anchor region 74 is disposed near proximal end 71, and link region 76 is disposed between body segment 73 and anchor region 74.

Body segment 73 generally includes longitudinal standards 78. Longitudinal standards confer columnar strength upon the device, and importantly to the positioning of the finished device, also comprise some flexibility. Longitudinal standards 78 are disposed in close proximity to one another near distal end 72 to define split 79. Split 79 imparts preferential bending and vessel lumen compatibility to the body of a finished device. Longitudinal standards 78 diverge from one another at the proximal end of split 79, and continue to diverge until they are positioned apart at the proximal end of body segment 73. Longitudinal standards 78 thereby define gap 80 with link region 76. In a finished device, gap 80 forms an open side of the device between the body and the anchor. As described in further detail below in relation to FIGS. 6A-7B, the open side will typically be positioned at or near the bifurcation “intersection” between the main vessel and the branch vessel, to accommodate blood flow through the vessel.

Throughout much, if not the full length body segment 73, and extending from longitudinal standards 78, are struts 82. Struts 82 are generally parallel to one another and vary in length from longest at the distal end 72, and shortest near link region 76. Struts 82 include apexes 84. Apexes 84 typically impart flexibility to struts 82. Prior to deployment of the device, most of the stress imparted on the device occurs during crimping down and loading the device into a sheath, and then tracking the crimped device through tortuous vasculature while crimped down and sheathed. Following deployment of the device within the vessel, most of the stress imparted on the device is a result of the ongoing, long term expansion and contraction due to pulsation of the vessel. In both configurations, the majority of the stress on the device is absorbed by the apexes 84. It is desirable for the device to be able to flex at the apexes 84; otherwise the stress may break the device, or deform the device beyond its ability to recover, or otherwise cause the device to fail.

At the same time, somewhat close spacing between struts 82 must be maintained in order for diversion of blood flow to be achieved. Further, consistent spacing between struts is important for facilitating loading and unloading of a finished device. In order to accommodate these requirements, disposed near apexes 84 are S-connectors 86. S-connectors 86 limit gaping between struts 82 by imparting some rigidity to struts 82. Close spacing between struts 82 assist high coverage region 15 in diverting blood flow from entering an aneurysm and also in providing the support for embolic coils. In addition, loading and unloading is facilitated by consistent spacing between struts 82. S-connectors 86 limit gaping without sacrificing needed flexibility of struts 82. S-connectors 86 are strategically disposed a short distance from apexes 84, rather than directly upon apexes 84. Consequently, apexes 84 are permitted flexure throughout the life of the device, while sufficient close spacing is maintained. Apexes 84 additionally point in the direction of distal end 93, facilitating resheathing of the device if needed.

Longitudinal standards 78 extend proximally from body segment 73 and throughout much, if not all, of link region 76. In link region 76, longitudinal standards 78 do not have struts, and thus are the only structure defining link region 76. (In the pattern 70, longitudinal standards 78 have been cut according to pattern 70, but have not yet been curved and shape set to define the hairpin turns 40 illustrated in FIG. 4A.) Longitudinal standards 78 continue proximally, and divide to define tines 88 and top apexes 89 of cells 90a. Tines 88 diverge to a widest apart position, and then turn towards one another, to define lateral apexes 92 of cells 90a. Tines 88 eventually rejoin to define bottom apexes 93. Tines 88 then again divide, diverge to a widest point, and turn toward one another to form lateral apexes 94 of cells 90c. Tines 88 eventually rejoin to define bottom apexes 95. Bottom apexes 95 extend proximally to form proximal extensions 96. Proximal extensions 96 may be in the form of eyelets or any structure which can accommodate radiopaque markers. Alternative embodiments may include loops, hooks, tabs, or comparable structure, and a marker or markers disposed in other locations, such as, for example, at the distal end of the device. The proximal extensions 96 are between 0.010-0.050 inch, and preferably between 0.015 and 0.030 inch in length. Some variations of the foregoing pattern are within the scope of the invention. A comparable pattern may be used to cut a tubular structure to manufacture a device according to the invention.

Returning now to a description of the loading, delivery and deployment of finished devices, a bridge device that is part of a system according to the invention may be delivered via minimally invasive techniques to the site of a cerebral aneurysm. An early step in such a method includes crimping a first bridge device to a reduced profile configuration, and then loading the bridge device into a suitable delivery catheter. It is retained in a reduced profile delivery configuration by a sheath, and it is capable of expanding into contact with the vessel walls when released or deployed to a larger diameter configuration. A suitable sheath may be an elongate tubular catheter formed of a polymeric material such as Pebax nylon, urethane, PTFE, Polyimide, metals such as Stainless Steel, Platinum, etc., or other suitable material. The suitable sheath is proportioned for passage through cerebral vasculature, and may have an outer diameter in the range of 1 mm-3 mm. A device remains in the reduced profile configuration during tracking of the device under fluoroscopic visualization to a treatment site within the vasculature of a subject.

A method according to the invention illustrated beginning in FIG. 6A. Bridge device 100 has been crimped to a reduced profile configuration and is retained by sheath 102, which has been loaded into an elongate delivery device such as delivery catheter 103. A guide wire (not pictured) may be utilized in the step of positioning the system in a vessel. First bridge device 100 is tracked under fluoroscopic visualization across turn T in order to place body 120 within left branch vessel L. In this early step of a method according to the invention, first bridge device 100 is placed slightly distally to the desired final position of the device. As illustrated in FIG. 6B, proximal anchor portion 124, (which is inside sheath 102 and will be deployed in a later step within vessel V), is now positioned slightly distally of parent vessel V.

In order to deploy first bridge device 100, sheath 102 is withdrawn proximally enough to permit body 120 of first device 100 to expand to its unconstrained configuration into contact with the inner walls of left branch vessel L. (See FIG. 6C.) Body 120 has struts 125. Though it is not readily visible in FIG. 6C, body 120 has a split and an open side that does not include struts, as illustrated in devices of previous figures. Once most of body 120 has expanded, but before sheath 102 is withdrawn sufficiently to release anchor 124, delivery catheter 103, and consequently bridge device 100, is retracted a small distance proximally within the vessel. The process of retracting delivery catheter 103 proximally causes bridge device 100 to cross the turn T between left branch vessel L and parent vessel V. Because the split and the gap (or open side) of a finished device bends preferentially over the portions of body 120 that have struts, the process of retracting the system causes bridge device 100 to self-position the gap along the bend. The gap positions along the bend facing vessel V. Consequently, the opposite side of body 120, bearing struts, is disposed along neck N of aneurysm A. Delivery catheter 103 is retracted proximally until anchor 124 is positioned within vessel V.

Sheath (catheter) 102 is withdrawn further to permit proximal anchor 124 to expand to its unconstrained configuration into contact with the inner walls of parent vessel V. When sheath (catheter) 102 is partially retracted to unsheathe the distal section of first device 100, the distal section of the device expands to a deployed diameter inside the branch vessel, as illustrated in FIG. 6E. If the user wishes to reposition first device 100, sheath (catheter) 102 can be advanced distally in order to resheath first device 100. The steps of positioning and deploying first device 100 described above can then be repeated. When first device 100 is deployed to the satisfaction of the operator, sheath 102 and delivery catheter 103 or other elongate delivery device can be withdrawn from the vessel.

Turning now to FIG. 7A, a coil delivery catheter 128 may be tracked through open portions of anchor 124 and bridge 120 and into aneurysm A. Embolic coil 129 or other desired embolic device and/or material may be delivered through catheter 128 and into aneurysm A. In some cases, the clinician may determine that a single bridge device and a single coil are sufficient to achieve treatment objectives. In other cases, additional coils may be desired. And in still other cases, the clinician may determine that a second bridge device is desired to maximize coverage of the neck of the aneurysm.

In cases in which a second bridge device is desired steps that are similar to the preparation and deployment of first bridge 120 are followed with respect to second bridge 130. Specifically, second bridge 130 is crimped into a delivery configuration and retained by a sheath 132, and loaded into a delivery catheter 133. Following steps similar to those outlined above for deployment of first bridge device 100, second bridge device 130 is tracked to the site of the aneurysm using fluoroscopic visualization. Second device 130 tracked through the interior of deployed proximal anchor 124, and further into right branch vessel R. Body 134 is positioned distally of its desired final position within right branch vessel R. Sheath 132 is partially withdrawn to permit body 138 to expand. Delivery catheter 133 is then retracted proximally, and bridge device 130 bends preferentially along its open side, causing bridge device 130 to orient itself such that the open side of bridge 130 faces vessel V, and the struts face neck N. And consequently, some of struts 136 of bridge 130 are positioned to overlap some of struts 125. The overlap results in a high coverage region of the system across the neck N. Sheath (catheter) 132 is then withdrawn proximally, as shown in FIG. 7B, to permit anchor 134 to expand within anchor 124 and into contact with the walls of vessel V. When second device 130 is positioned and deployed to the satisfaction of the operator, sheath (catheter) 132 can be withdrawn from the vessel, and system 200 may remain either temporarily or permanently implanted in the vessel. (See FIG. 7B.)

As mentioned above, a system according to the invention may be shaped, sized, and configured in numerous ways in order to accommodate particular vessel morphology. A few examples of variations are illustrated in FIGS. 8A-8D. The examples demonstrate the devices', and consequently the system's adaptability to widely varied vessel geometries or “angio archictectures”. This versatility is the result of the great flexibility in the links between the spans and the anchors of the bridge devices, enabling the “T” span to accommodate an extremely wide range of angles at which vessels may branch.

System 140 is configured so the first device 150 is similar to the example described above, but second device 160 is configured such that body 162 is oriented at a steeper angle to body 152. Flexibility in links 158 and 168 and the range of possible shape sets enable a range of configurations of system 140. In the example of system 170, which is illustrated in FIG. 8B, both body 182 and body 192 are oriented at a tight angle to one another Links 188 and 198 are generally of the same shape in the example of FIG. 8B. In FIG. 8C, body 212 of system 200 is generally 180 degrees to body 222. But body 212 is oriented at a tight angle with respect to proximal anchor 214, and body 222 is oriented to proximal anchor 224 at a very wide angle Links 217 and 227 are of very different shapes from one another in the example of FIG. 8C. And in FIG. 8D, both body 242 and body 252 are oriented at acute angles to proximal anchor 244 and proximal anchor 254. The foregoing are merely a few examples of a very wide array of configurations (or angio architecture) that are within the scope of the invention.

It should be recognized that a number of variations of the above-identified embodiments will be obvious to one of ordinary skill in the art in view of the foregoing description. Accordingly, the invention is not to be limited to those specific embodiments and methods of the present invention illustrated and described herein. Rather, the scope of the invention is to be defined by the claims and their equivalents.

What is claimed is:

1. A system for use in treatment of an aneurysm having a neck, the aneurysm located near a bifurcation in a vessel, the system comprising:
at least one elongate delivery device; and
at least one intravascular device comprising a reduced profile delivery configuration and an expanded profile deployment configuration, a proximal end and a distal end, a generally tubular segment having a longitudinal gap, the generally tubular segment disposed near the distal end and positionable across the neck of the aneurysm; an anchor disposed near the proximal end and positionable within the vessel; and a flexible link joining the generally tubular segment and the anchor, the flexible link positionable within or across a bifurcation in a vessel.

2. The system according to claim 1 wherein said longitudinal gap extends the length of the generally tubular segment.

3. The system according to claim 2 wherein said longitudinal gap has a first width at the proximal end of the generally tubular segment and a second width at the distal end of the generally tubular segment, wherein said first width is greater than said second width.

4. The system according to claim 1 wherein said generally tubular segment comprises at least one longitudinal standard and a plurality of struts extending a length from said longitudinal standard to define a generally tubular wall, wherein said struts terminate to define the longitudinal gap in the wall.

5. The system according to claim 1 wherein said generally tubular segment comprises a first longitudinal standard and a second longitudinal standard and struts extending from the first longitudinal standard to the second longitudinal standard, wherein said longitudinal gap is defined by the open space between the first longitudinal standard and the second longitudinal standard.

6. The system according to claim 5 wherein said struts are generally V-shaped.

7. The system according to claim 6 wherein said V-shaped struts have an apex and further comprise S-shaped members disposed near the apex.

8. The system according to claim 1 wherein said anchor comprises a first and a second lateral apex for engaging the wall of the vessel.

9. The system according to claim 5 wherein said first and second longitudinal standards extend proximally from said generally tubular element to said define said flexible link, and wherein said first longitudinal standard further splits to form a first and a second tine, and each tine extends proximally; and said second longitudinal standard further splits to form a first and a second tine, and each tine extends proximally, each tine of the first longitudinal standard then converging with each tine of said second longitudinal standard to define lateral apexes of the anchor.

10. The system according to claim 1 wherein said system further comprises an embolic device or material positionable through said gap and into the aneurysm.

11. A system for use in treatment of an aneurysm having a neck, the aneurysm located near a bifurcation in a vessel, the system comprising:
at least one elongate delivery device; and
a first intravascular device and a second intravascular device, at least one of the first and second intravascular devices comprising a reduced profile delivery configuration and an expanded profile deployment configuration, a proximal end and a distal end, a generally tubular segment disposed near the distal end and positionable across the neck of the aneurysm, an anchor disposed near the proximal end and positionable within the vessel, and at least one flexible link joining the generally tubular segment and the anchor, the flexible link positionable within or across a bifurcation in a vessel; wherein said generally tubular segment comprises a longitudinal gap.

12. The system according to claim 11 wherein said longitudinal gap extends the length of the generally tubular segment.

13. The system according to claim 12 wherein said longitudinal gap has a first width at the proximal end of the generally tubular segment and a second width at the distal end of the generally tubular segment, wherein said first width is greater than said second width.

14. The system according to claim 11 wherein said generally tubular segment comprises at least one longitudinal standard and a plurality of struts extending a length from said longitudinal standard to define a generally tubular wall, wherein said struts terminate to define the longitudinal gap in the wall.

15. The system according to claim 11 wherein said generally tubular segment comprises a first longitudinal standard and a second longitudinal standard and struts extending from the first longitudinal standard to the second longitudinal standard, wherein said longitudinal gap is defined by the open space between the first longitudinal standard and the second longitudinal standard.

16. The system according to claim 15 wherein said struts are generally V-shaped.

17. The system according to claim 16 wherein said V-shaped struts have an apex and further comprise S-shaped members disposed near the apex.

18. The system according to claim 11 wherein said anchor comprises a first and a second lateral apex for engaging the wall of the vessel.

19. The system according to claim 15 wherein said first and second longitudinal standards extend proximally from said generally tubular element to said define said flexible link, and wherein said first longitudinal standard further splits to form a first and a second tine, and each tine extends proximally; and said second longitudinal standard further splits to form a first and a second tine, and each tine extends proximally, each tine of the first longitudinal standard then converging with each tine of said second longitudinal standard to define lateral apexes of the anchor.

20. The system according to claim 11 wherein said system further comprises an embolic device or material positionable through said gap and into the aneurysm.

21. The system according to claim 15 wherein both the first and the second intravascular devices comprise struts and said system further comprises an area of overlap of said struts.

22. The system according to claim 21 wherein said area of overlap is positionable across the neck of the aneurysm.

23. A method of manufacture of an intravascular device, said method comprising the steps of:
providing a tube comprising one or more shape memory materials;
cutting the tube according to a predetermined pattern to impart a generally tubular segment having a plurality of struts and a longitudinal gap between the struts; an anchor and a link between the generally tubular segment and the anchor;
shaping the intravascular device to impart a desired orientation of the generally tubular segment to the anchor;
applying a shape memory set to the shaped cut tube.

24. The method according to claim 23 wherein said shape memory material is nitinol.

25. The method according to claim 23 wherein said predetermined pattern defines at least two elongate standards.

26. The method according to claim 25 wherein said predetermined pattern defines the plurality of struts having at least one end connected to an elongate standard.

27. The method according to claim 23 wherein said struts are V-shaped.

28. A method of treating an aneurysm located in a blood vessel of a subject, the aneurysm having a neck and the method comprising the steps of:
introducing into the blood vessel a first intravascular device that is radially expandable from a compressed position to an expanded position, the intravascular device comprising a tubular element having a longitudinal gap; an anchor; and a link between the tubular element and the anchor;
bridging at least some of the neck of the aneurysm with the tubular element;
deploying the anchor within the vessel; and
delivering one or more embolic materials to the aneurysm.

29. The method according to claim 28 wherein the step of bridging the neck of the aneurysm includes introducing the tubular element distally of the aneurysm;
partially deploying the intravascular device so that the tubular element is expanded; then retracting the intravascular device proximally to permit the tubular element to align its struts along the neck of the aneurysm and to align the longitudinal gap opposite the aneurysm; and
then fully deploying the intravascular device to permit the anchor to engage the vessel wall.

30. The method according to claim 28 with the additional step of deploying a second intravascular device prior to the step of delivering an embolic material, by introducing and deploying the second intravascular device through the anchor of the first intravascular device.

31. The method according to claim 30 wherein each tubular element has a plurality of struts and said second intravascular device is deployed so that the struts of each tubular element overlap to form a high coverage region across at least some of the aneurysm neck.

32. The method according to claim 31 wherein the step of deploying said second intravascular device further comprises introducing said second intravascular device into the vessel distally of the aneurysm; partially deploying said second intravascular device so that the tubular element is expanded; retracting the intravascular device proximally to permit the struts to self align with the neck of the aneurysm and the longitudinal gap to self align opposite the aneurysm neck; deploying the entire intravascular device to permit the anchor to engage the vessel wall.
